(19) United States
(12) Patent Application Publication
(10) Pub. No.: US 2023/0050224 A1
(43) Pub. Date: Feb. 16, 2023

(54) ORTHOPEDIC SCREWS AND PROSTHESES FOR USE WITH POLYMER CABLES

(71) Applicant: DSM IP ASSETS B.V., Heerlen (NL)

(72) Inventors: Brian WOODALL, Exton, PA (US); Gerald WARE, Exton, PA (US)

(21) Appl. No.: 17/784,358

(22) PCT Filed: Dec. 18, 2020
(86) PCT No.: PCT/US2020/065784

§ 371 Date: Jun. 10, 2022

Related U.S. Application Data
(60) Provisional application No. 62/986,195, filed on Mar. 6, 2020.
(60) Provisional application No. 62/949,957, filed on Dec. 18, 2019.

(51) Int. Cl. A61B 17/70

(52) U.S. Cl. CPC A61B 17/7032; A61B 17/7035

(57) ABSTRACT

Orthopedic screws and prostheses for use with polymer cables are disclosed herein. An orthopedic screw is configured to retain a polymer cable (12) when the polymer cable is positioned in a thru hole (5) present in the body of said orthopedic screw. This may be accomplished by an orthopedic screw comprising a set screw and a complimentary threaded hole, wherein the threaded hole intersects the thru hole and wherein the polymer cable is secured in the thru hole by tightening the set screw. In an embodiment, the threaded hole extends beyond the thru hole.

### CROSS-REFERENCE TO RELATED APPLICATIONS

This application is an International Application claiming priority to U.S. Provisional Application No. 62/949,957, filed 18 Dec. 2019, and U.S. Provisional Application No. 62/986,195, filed 6 Mar. 2020, the entire contents of each of which are hereby incorporated by reference in their entirety.

### FIELD

The inventions relate to screws and prostheses for orthopedic stabilization. Such screws may be inserted into bone, such as into a vertebra through a pedicle area of the vertebra, in order to fix an area of the skeletal anatomy of a patient.

### BACKGROUND

Orthopedic screws are commonly used to fix a patient's anatomy in place in order to allow for the anatomy to heal. Orthopedic screws are screwed into a patient's bone and may be used to fix two pieces of bone together, for instance in the case of a fracture, or to secure a prosthesis to one or more areas of anatomy.

Some orthopedic screws are designed to allow fixation of a prosthesis to the screws. Typically, one or more screws are secured to one or more bones and a prosthesis secured to the screws. The prosthesis may be a bone plate, wherein the screws secure the bone plate against a bone.

Other prostheses require screws that have separate mechanisms for fixing the prosthesis to a screw. Such screws typically comprise a threaded cradle wherein a portion of a prosthesis is secured via a nut or set screw that is securable to the threaded cradle. The threaded cradle may comprise a thread on its exterior, in the case the prosthesis is securable via a nut, or in its interior, in the case the prosthesis is securable via a set screw. For example, the prosthesis may comprise a metal rod that is placed in the cradle and secured in the cradle by tightening a set screw. Such rods may form all or part of a spinal prosthesis.

One challenge when securing a prosthesis to a pedicle screw is positioning of the prosthesis in the cradle of the screw. For example, a spinal stabilization rod must be inserted into the cradles of multiple orthopedic screws and secured in each orthopedic screw.

Orthopedic screws used for securing prostheses generally are either mono-axial or poly-axial. In a mono-axial screw the shaft and cradle are formed as one part such that the cradle cannot move relative to the shaft. In a poly-axial screw a joint is present between the shaft and the cradle such that the cradle may be angled relative to the shaft. Mono-axial screws possess greater strength and often a lower profile over poly-axial screws. Poly-axial screws are easier to align to a prosthesis, but the greater profile may make it difficult or impossible to place them at adjacent levels in many lordotic spines.

An important design criterion of orthopedic screws is reducing the profile of the screw. A lower profile screw increases patient comfort and reduces complications.

### SUMMARY

The inventors have realized that an orthopedic screw can be configured to appropriately retain a polymer cable, enabling the use of polymer cables instead of or in addition to other prostheses. For example, the polymer cable may be directly attached to a patient's anatomy or be used to attach a prosthesis to the orthopedic screw.

Certain devices that allow for polymer cables to secure a portion of a patient's anatomy are known. For example, such devices are disclosed in U.S. Pat. No. 7,207,090, US2011/0270314, and WO2019/055723. Another example of such a device is the BandLoc product by OrthoPediatrics®. The BandLoc product comprises a polyester tether that is secured in a cradle against a spinal prosthesis rod held in place by a set screw. The system requires traditional pedicle screws to secure the spinal prosthesis, via a threaded cradle and nut or set screw, at other locations along the spine.

In an embodiment, an orthopedic screw is configured to retain a polymer cable when the polymer cable is positioned in a thru hole present in the body of said orthopedic screw. This may be accomplished by an orthopedic screw comprising a set screw and a complimentary threaded hole, wherein the threaded hole intersects the thru hole and wherein the polymer cable is secured in the thru hole by tightening the set screw. In an embodiment, the threaded hole extends beyond the thru hole.

In accordance with an embodiment of the invention, a prosthesis is securable to an orthopedic screw with a polymer cable secured in the orthopedic screw. The polymer cable is secured to the body of an orthopedic screw by, in an embodiment, a set screw insertable into the body of the screw, thereby securing the polymer cable in the screw with little added profile. A prosthesis may then be secured in a cradle by fixing the polymer cable in place around the prosthesis. A threaded cradle typically used to fix a prosthesis to an orthopedic screw in the prior art adds significantly to the profile of the orthopedic screw. Securing a prosthesis to the orthopedic screw by other means could thus reduce the profile of an orthopedic screw, leading to improved patient comfort.

In an embodiment, an orthopedic screw comprises:
a. a screw body comprising
   i. a head;
   ii. a shank connected to the head and comprising an exterior thread, the shank having a longitudinal axis,
   iii. a thru hole extending from one side of the screw body to the other and through the longitudinal axis, and
   iv. a threaded hole intersecting the thru hole, and extending at least from an exterior surface of the head to past the thru hole, the threaded hole comprising a threaded hole end and a threaded hole thread, and
b. a set screw comprising a set screw thread, the set screw thread being complimentary to the threaded hole thread, and the set screw comprising a set screw end complimentary to the threaded hole end.

The inventions disclosed herein may have numerous benefits, such as improved screw strength, easier fixation of a prosthesis to a screw, improved costs, lower weight, lower profile, and easier screw placement in lordotic spines.

### BRIEF DESCRIPTION OF THE DRAWINGS

FIG. 1 is a side view of an embodiment of a screw body of an orthopedic screw.

FIG. 2 is a side view of an embodiment of a set screw for use with the screw body of FIG. 1.

FIG. 3 depicts an embodiment of an orthopedic screw comprising a set screw engaged in a screw body.

FIG. 4 depicts an embodiment of an orthopedic screw comprising a head in the shape of a rectangular prism.

FIG. 5 is a perspective view of an embodiment of an orthopedic screw retaining a polymer cable.

FIG. 6 depicts a plurality of orthopedic screws inserted into vertebrae of a patient and connected via polymer cables.

FIG. 7 depicts side views of an embodiment of an orthopedic screw comprising a cradle.

FIG. 8 depicts side views of a polymer cable securing a prosthesis in the cradle of an embodiment of an orthopedic screw.

FIG. 9 depicts a plurality of orthopedic screws inserted into vertebrae of a patient. Polymer cables are used to fix prostheses to the plurality of orthopedic screws.

FIG. 10 is a perspective view of an embodiment of a prosthesis.

FIG. 11 is a perspective view of an embodiment of a prosthesis.

FIG. 12 is a side view of an embodiment of a prosthesis.

FIG. 13 is a side view of a polymer cable securing a prosthesis in the cradle of an embodiment of an orthopedic screw.

FIG. 14 depicts side views of an embodiment of an orthopedic screw comprising a cradle.

FIG. 15 depicts a cross-section of a polymer cable securing a prosthesis in the cradle of an embodiment of an orthopedic screw.

FIG. 16 depicts a cross-section of a polymer cable securing an embodiment of a prosthesis in the cradle of an embodiment of an orthopedic screw.

FIG. 17 depicts a cross-section of a polymer cable securing a prosthesis in the cradle of an embodiment of an orthopedic screw.

FIG. 18 is a side view of Screw A of Example 1.

FIG. 19 is a side view of Screw B of Example 1.

FIG. 20 is a side view of Screw C of Example 1.

FIG. 21 is a plot of load vs. time associated with Screw A of Example 1.

FIG. 22 is a plot of load vs. time associated with Screw B of Example 1.

FIG. 23 is a plot of load vs. time associated with Screw C of Example 1.

### DETAILED DESCRIPTION

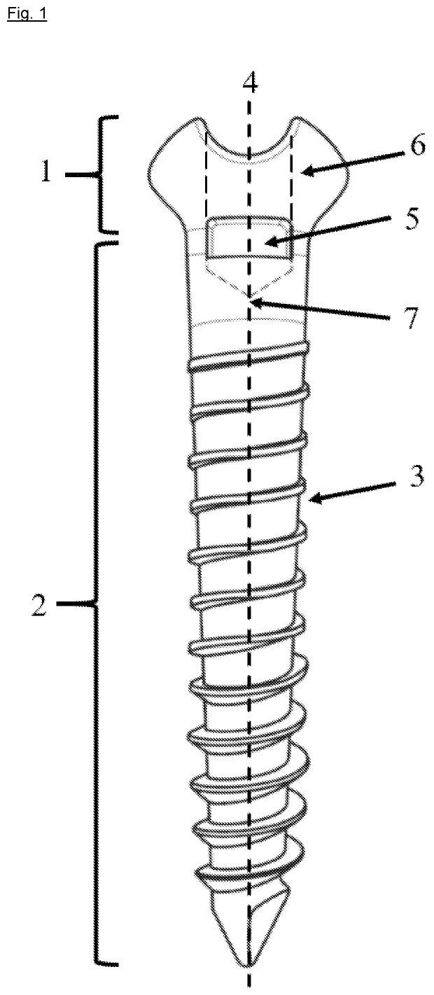

FIG. 1 depicts the screw body of an embodiment of an orthopedic screw. The screw body comprises a head 1 and a shank 2. The shank comprises an exterior thread 3. The exterior thread may be configured to secure the screw in a bone of a patient, such as the pedicle area of a vertebra. The exterior thread may have any useful thread design, such as a thread having a uniform pitch or a plurality of pitches. The screw possesses a longitudinal axis 4 extending down its center.

The screw body comprises a thru hole 5 extending from one side of the screw body to the other and through the longitudinal axis. The thru hole 5 extends through the screw from one side to the other and may intersect the longitudinal axis 4. In an embodiment, the thru hole comprises a central axis transverse to the longitudinal axis 4. The thru hole 5 is adapted to receive a polymer cable. The thru hole can be any suitable shape. In an embodiment, the thru hole has a shape complimentary to the polymer cable. In an embodiment, the thru hole is circular, rectangular, or trapezoidal.

The orthopedic screw comprises a threaded hole 6 interior to the screw body and indicated by the dashed line. The threaded hole 6 extends at least from an exterior surface of the head 1 to past the thru hole 5 in the direction of the longitudinal axis 4. In an embodiment, the center axis of the thru hole is orthogonal to the center axis of the threaded hole. The threaded hole 6 comprises an interior thread. The threaded hole comprises a threaded hole end 7. In an embodiment, the threaded hole is complimentary in geometry and threading to a set screw. In an embodiment, the threaded hole does not extend completely through the screw body. In an embodiment, the threaded hole is a blind hole.

In an embodiment, the orthopedic screw is a mono-axial screw. In an embodiment, the orthopedic screw is a poly-axial screw. A poly-axial screw differs from a mono-axial screw in that the head of the screw may move relative to the shank, such as by means of a ball joint. In an embodiment, the head and the shank are formed as a continuous, single piece.

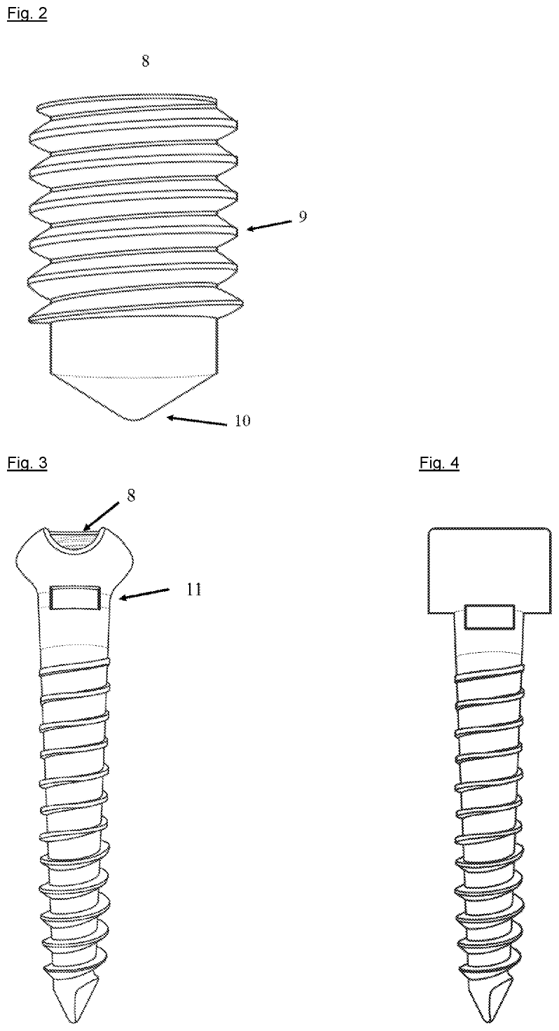

FIG. 2 depicts the set screw 8 in detail. The set screw comprises a set screw thread 9 that is complimentary to the threaded hole thread. In an embodiment, the set screw thread 9 is present over less than the entire length of the set screw. The set screw 8 comprises a set screw end 10 that is complimentary to the threaded hole end 7. Preferably, there is no clearance between the threaded hole end and the set screw end when the set screw is fully inserted in the threaded hole. In an embodiment, the set screw end is conical. In an embodiment, the set screw end is frustoconical. In an embodiment, the set screw end is flat. In an embodiment, the set screw end is a portion of a spheroid. The threaded hole end is complimentary in shape. The set screw may be insertable with a standard screwdriver and can take any suitable screw drive format.

FIG. 3 depicts the orthopedic screw with the set screw 8 engaged in the threaded hole. After full insertion, the top of the set screw preferably does not extend farther than the top of the head of the screw body 11. In an embodiment, and as depicted, the set screw thread does not extend to the thru hole. In an embodiment, the set screw thread is visible in the thru hole upon full insertion of the set screw in the threaded hole.

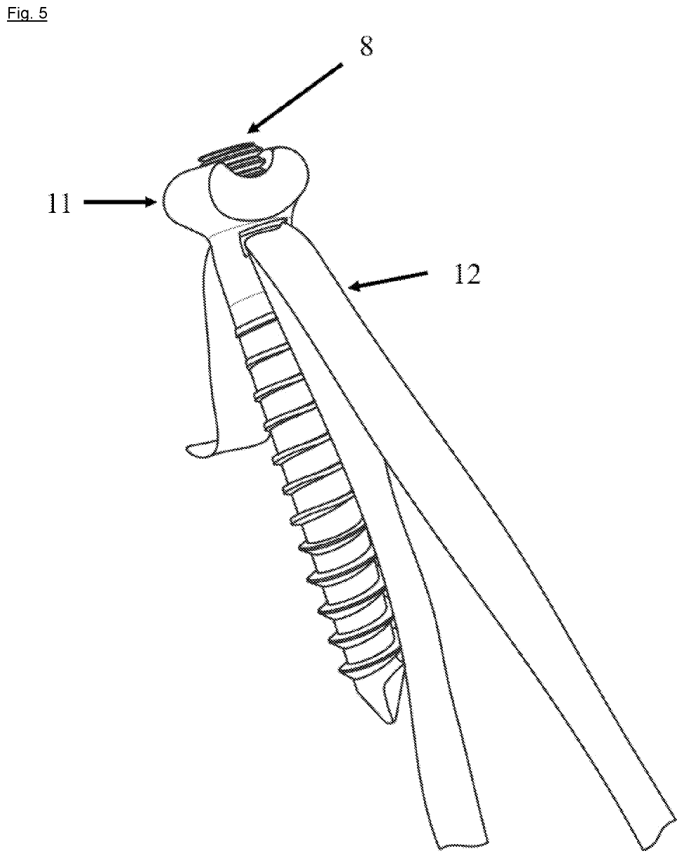

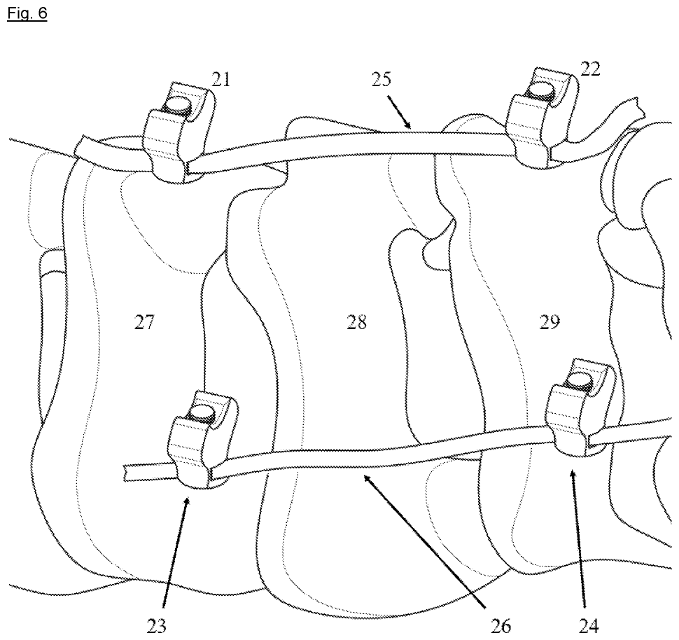

The set screw head may have any suitable shape. For example, the set screw head may be a square or rectangular prism, as depicted in FIG. 4.

In an embodiment, the orthopedic screw is configured to retain a polymer cable positioned in the thru hole, such as by securing the polymer cable. Securing a polymer cable means fixedly engaging a polymer capable such that the polymer cable will not slide in the thru hole under normal use conditions. FIG. 5 depicts such an embodiment. The depicted polymer cable 12 comprises a substantially rectangular cross-section complimentary to the shape of the thru hole. The polymer cable 12 is secured by placing one or more polymer cables in the thru hole and tightening the set screw 8. Upon tightening, the set screw end 8 moves proximate the threaded hole end and retains the polymer cable in the thru hole. In an embodiment, the threaded hole end extends past the thru hole such that the polymer cable does not pass straight through the thru hole when the set screw is fully engaged. Although the set screw 8 is depicted as rising above the head of the orthopedic screw 11 for convenience, ideally the set screw does not protrude above the head of the orthopedic screw once it is fully inserted into the threaded hole. In an embodiment, the set screw does not protrude above the head of the orthopedic screw once the set screw is fully tightened.

A polymer cable is a cable comprised of one or more polymers. In an embodiment, the polymer cable comprises polyethylene. In an embodiment, the polymer cable comprises polymer fibers. A fiber is a long continuous filament. In an embodiment, the polymer cable consists of polymer fibers. In an embodiment, the polymer cable comprises a braid of a plurality of strands of individual polymer cables or fibers. In an embodiment, the polymer cable comprises a braid that comprises a hollow tubular braid, a solid circular braid, a spiroid braid, a flat braid, a core-sheath (sometimes called kern-mantle or core-shell) braid, or a braid-on-braid. A braid-on-braid is a core-shell construction wherein a braided core is covered by another braided construction. In an embodiment, the polymer cable comprises a yarn. A yarn is a continuous strand of multiple, usually twisted, fibers. In an embodiment, the polymer cable comprises a braided, knitted, or woven cable, wherein the polymer cable comprises polymer fibers. In an embodiment, the polymer cable comprises a monofilament or a multifilament yarn. The yarn may in addition comprise other components or additives that provide some extra functional effect, such as antimicrobial or anti-inflammatory action, knotting performance, or visual contrast. In an embodiment, the polymer cable comprises a radiopaque agent.

In an embodiment, the set screw end comprises a piercing element arranged to pierce a polymer cable present in the thru hole. Piercing means that a portion of the polymer cable is penetrated, for instance if the piercing element possesses a smaller cross-section than the diameter of the polymer cable and penetrates through the polymer cable, or that the piercing element is able to pass through the body of the polymer cable without penetrating it, or some combination of the two. For instance, if the piercing element separates the strands of a braided cable or yarn and passes between the strands of the braided cable or yarn without penetrating or severing the strands of the polymer cable then the polymer cable is penetrated. Preferably, the piercing element does not sever any portion of the polymer cable, though the invention may still be operable if one or more strands of a braid are severed when engaging the piercing element. The piercing element may cut or sever a sheath of the cable that surrounds an interior cable, such as a braid of polymer fibers, and then pass between the strands of the braid.

The piercing element may generally take a narrow, elongate shape that may penetrate a polymer cable arranged in the thru hole. In an embodiment, the set screw end comprises a plurality of piercing elements arranged to pierce a polymer cable present in the thru hole. In an embodiment, the threaded hole end comprises a receptacle for a piercing element present on the set screw end.

The orthopedic screw may be a pedicle screw. The orthopedic screw may be insertable into the pedicle area of a vertebra of a patient. In an embodiment, a plurality of orthopedic screws are connected by means of a polymer cable. FIG. 6 depicts vertebrae 27, 28, 29 of a patient. A plurality of orthopedic screws 21, 22, 23, 24 inserted into the pedicle area of two of a patient's vertebrae 27 and 29. Polymer cables 25, 26 are retained in the thru hole of each pedicle screw and secured by tightening a first set screw. Orthopedic screws 21 and 23 are inserted into a first vertebra 27. Orthopedic screws 22 and 24 ae inserted into a second vertebra 29.

In an embodiment, a first orthopedic screw and a second orthopedic screw are inserted into the bone of a patient. A polymer cable is secured in the thru hole of the first orthopedic screw by tightening the set screw. The polymer cable may be secured in the thru hole of the first orthopedic screw before or after insertion of the first orthopedic screw into the bone of the patient. The polymer cable is threaded positioned in the thru hole of the second orthopedic screw. A specific tension for the polymer cable or distance between the first and second orthopedic screws may be prescribed. Upon reaching the desired tension or distance, the set screw of the second orthopedic screw is tightened, thereby retaining the polymer cable in the second orthopedic screw.

In an embodiment, an orthopedic screw comprises a cradle at its head. The cradle may be used to fix a prosthesis to the orthopedic screw, such as a spinal prosthesis. The shape of the cradle may correspond to the shape of the prosthesis.

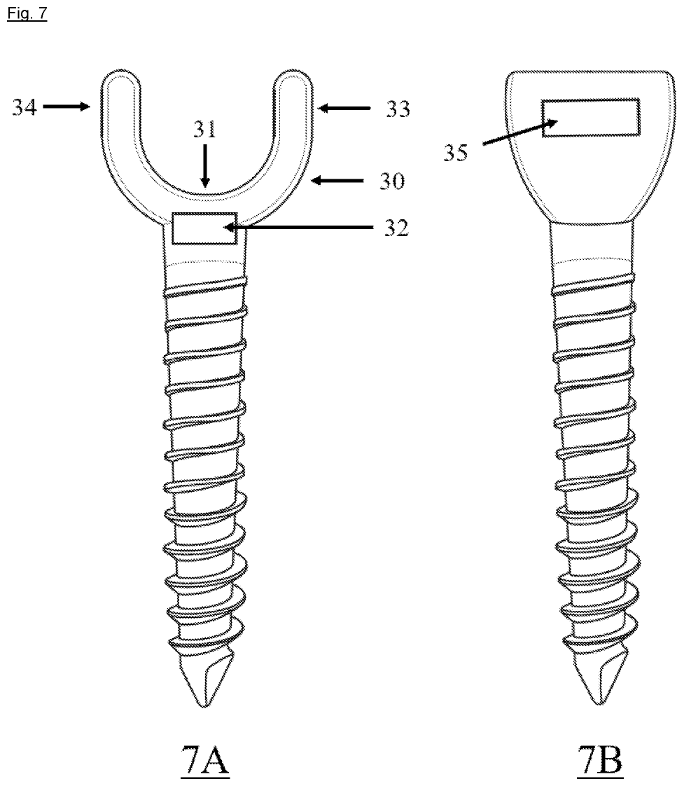

An orthopedic screw comprising a cradle is depicted in FIG. 7. FIG. 7A and FIG. 7B differ in that the orthopedic screw is rotated 90 degrees on its longitudinal axis. The depicted orthopedic screw comprises a head and a shank. The shank comprises an exterior thread. The head comprises a cradle 30. The cradle comprises two walls 33 and 34. The cradle is open at its top. The cradle is shaped to contain a spinal prosthesis having a circular cross section. Accordingly, the inner surface 31 of the cradle 30 has a semi-circular profile to correspond to the circular cross section of a spinal prosthesis. The cradle 30 is therefore adapted to support a cylindrical elongate member. The orthopedic screw comprises a thru hole 32 for receiving a polymer cable. The orthopedic screw may optionally comprise a set screw to secure a polymer cable in the first thru hole 32, for example, as depicted in the orthopedic screw depicted in FIG. 3.

The cradle comprises a second thru hole 35. As depicted in FIG. 7, the second thru hole is a wall thru hole. The second thru hole 35 can be used for securing a prosthesis in the cradle. For example, the polymer cable can be first secured in the first thru hole 32 and then through the second thru hole 35. The prosthesis can be tied down by knotting the polymer cable or attaching the polymer cable to the prosthesis. Although thru hole 32 and second thru hole 35 are depicted as having perpendicular axes in FIG. 7, it is possible that the thru hole 32 and second thru hole 35 have approximately parallel axes.

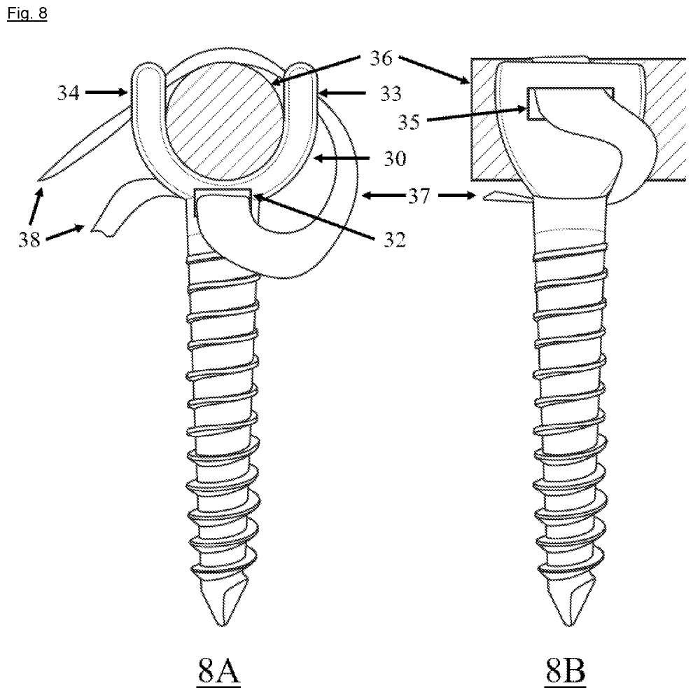

FIG. 8 depicts a circular prosthesis 36 arranged in cradle 30. FIGS. 8A and 8B differ in that the orthopedic screw is rotated 90 degrees on its longitudinal axis. The prosthesis 36 comprises a circular cross-section and corresponds with the semi-circular shaped inner surface of the cradle 30. A polymer cable 37 is secured in thru hole 32 by a set screw (not pictured). The spinal prosthesis 36 is inserted into cradle 30. The polymer cable 37 is placed in the portion of the second thru hole 35 present in wall 33, placed over the spinal prosthesis 36 and through the thru hole in the second wall 34. After appropriately tightening the polymer cable 37, the polymer cable may be secured by tying a knot or otherwise securing ends 38.

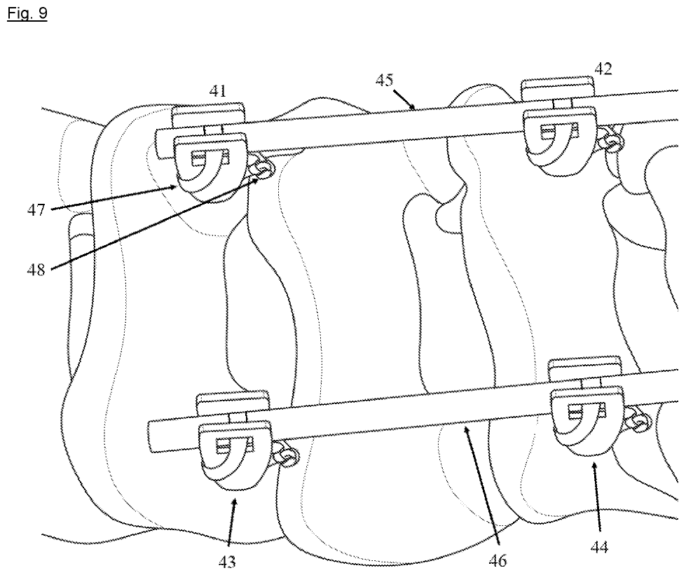

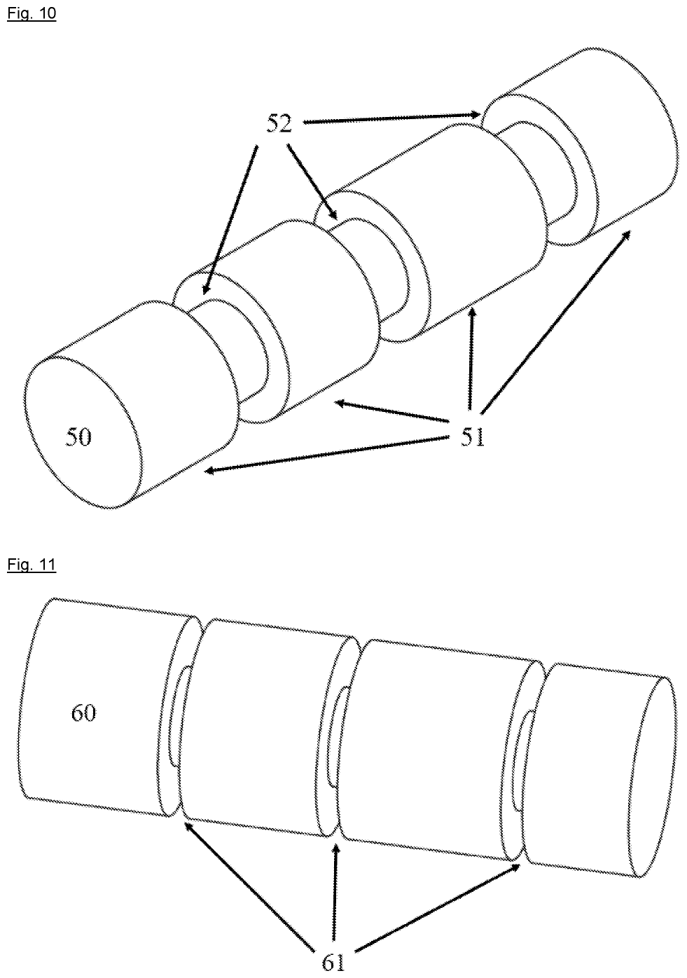

FIG. 9 depicts vertebrae a plurality of orthopedic screws 41, 42, 43, 44 inserted into the pedicle area of two of a patient's vertebrae. Polymer cables are retained in the thru hole of each screw and secured by tightening a first set screw. A first spinal prosthesis 45 is placed in the cradles of orthopedic screws 41 and 42. A second spinal prosthesis 46 is placed in the cradles of orthopedic screws 43 and 44. Spinal prosthesis 45 is secured to orthopedic screw 41 by means of polymer cable 47. Polymer cable 47 is passed through a first side of second thru hole 48, over the first spinal prosthesis 45, and through the second side of second thru hole 48. The polymer cable 47 is tightened and the spinal prosthesis 45 secured with a knot 49. The first spinal prosthesis 45 is secured similarly to orthopedic screw 42.

It is possible that the first spinal prosthesis 45 or second spinal prosthesis 46 experiences undesirable axial translation due to slipping of a polymer cable against the surface of the prosthesis. This may be mitigated by using a prosthesis comprising grooves, as depicted in FIG. 10. Prosthesis 50 possesses ridges 51 and grooves 52. A polymer cable may be positioned in one or more grooves 52 when securing the prosthesis to one or more orthopedic screws. In an embodiment, the dimension of a groove corresponds to the dimension of the polymer cable being used to secure the prosthesis.

A similar prosthesis is shown in FIG. 11. The prosthesis 60 comprises grooves wherein a polymer cable having a rectangular cross-section may be inserted into one or more grooves 61 by inserting the rectangular polymer cable leading with its minor dimension. A polymer cable may be looped through multiple grooves around the prosthesis in order to secure the prosthesis to the orthopedic screw.

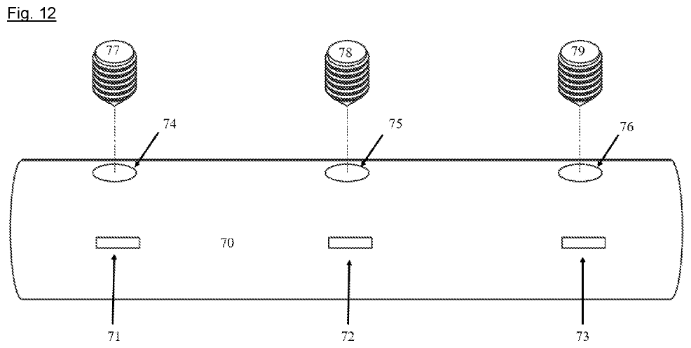

A further prosthesis is shown in FIG. 12. The prosthesis 70 comprises one or more prosthesis thru holes 71, 72, 73 through which a polymer cable may be passed. In an embodiment, the prosthesis comprises one or more prosthesis threaded holes, 74, 75, 76 and corresponding prosthesis set screws, 77, 78, 79. The prosthesis threaded holes and prosthesis set screws may have any suitable feature of the threaded hole and set screws of the orthopedic screws described previously, such as corresponding prosthesis threaded hole ends and prosthesis set screw ends, and a prosthesis threaded hole end that extends beyond the prosthesis thru hole.

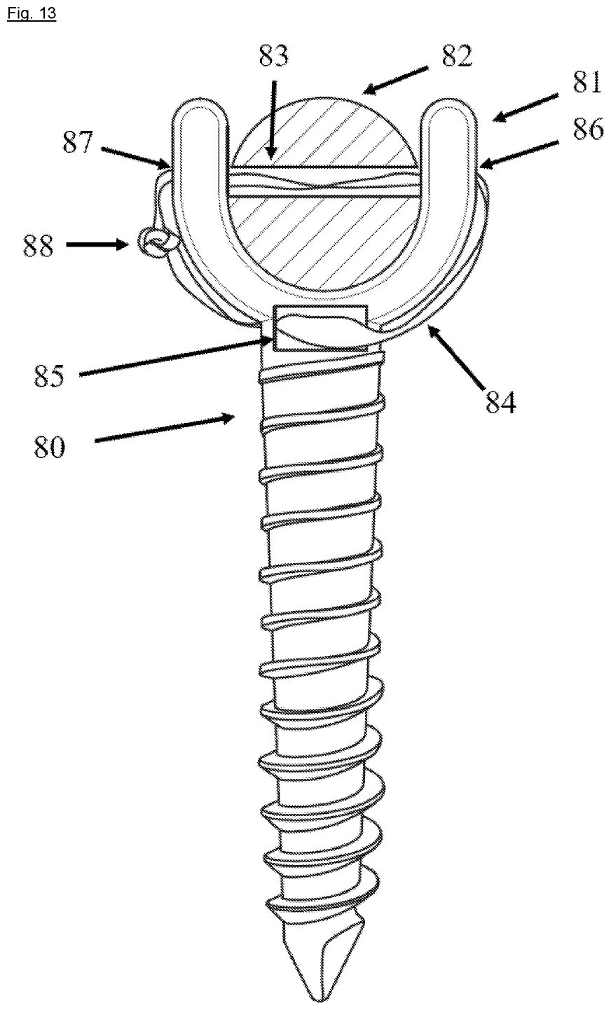

The prosthesis of FIG. 12 may be secured to an orthopedic screw, for example, the orthopedic screw of FIG. 7, as shown in FIG. 13. Orthopedic screw 80 comprises a cradle 81 in which sits prosthesis 82 comprising prosthesis thru hole 83. The prosthesis may possess a prosthesis threaded hole and a prosthesis set screw. Polymer cable 84 is secured in thru hole 85 by a set screw (not pictured). Polymer cable 84 is inserted into a first side of second thru hole 86, through prosthesis thru hole 83, and thru a second side of second thru hole 87. The polymer cable 84 is secured to the prosthesis 82 by knot 88. Alternatively, or in addition, polymer cable 84 could be secured to prosthesis thru hole 83 via a prosthesis threaded hole and prosthesis set screw. The knot may be obviated by passing both ends of the polymer cable through prosthesis thru hole 83 and tightening a prosthesis set screw in a prosthesis threaded hole. The excess length of polymer cable may then be trimmed.

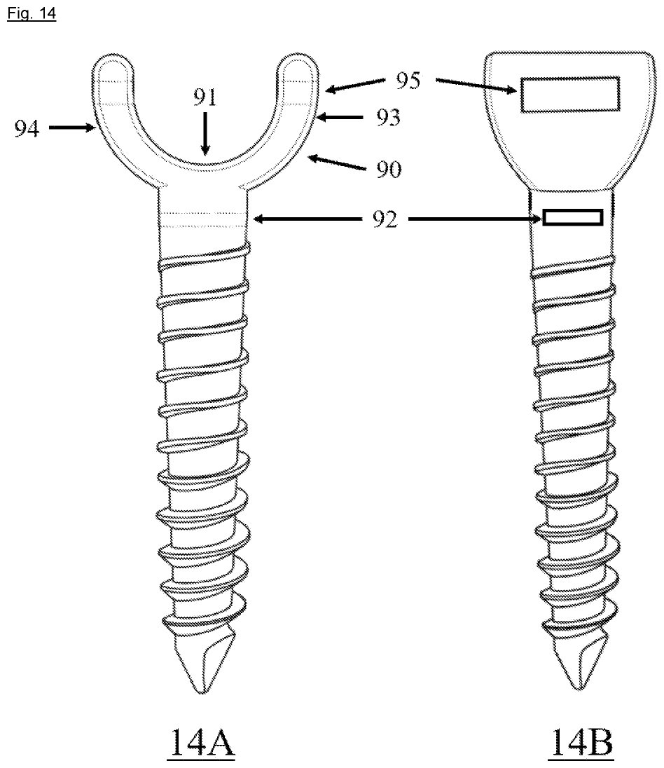

The prostheses of FIGS. 10, 11, and 12 may alternatively be secured to an orthopedic screw as shown in FIG. 14. FIG. 14A and FIG. 14B differ in that the view of the orthopedic screw is rotated 90 degrees on its longitudinal axis. The depicted orthopedic screw comprises a head and a shank. The shank comprises an exterior thread. The head comprises a cradle 90. The cradle comprises two walls 93 and 94. The cradle is open at its top. The depicted cradle is shaped to contain a spinal prosthesis having a circular cross section. Accordingly, the inner surface 91 of the cradle 90 has a semi-circular profile to correspond to the circular cross section of a spinal prosthesis. The cradle 90 is therefore adapted to support a cylindrical elongate member.

The orthopedic screw depicted in FIG. 14 comprises a thru hole 92 for receiving a polymer cable. The cradle comprises a second thru hole 95. The positions of first thru hole 92 and second thru hole 95 are depicted as dotted lines in FIG. 14A. As depicted in FIG. 14, the second thru hole is a wall thru hole. The second thru hole 95 can be used for securing a prosthesis in the cradle. For example, the polymer cable can be first secured in the first thru hole 92 and then through the second thru hole 95. The prosthesis can be tied down by knotting the polymer cable or attaching the polymer cable to the prosthesis. First thru hole 92 and second thru hole 95 may have parallel or approximately parallel axes. The orthopedic screw may optionally comprise a set screw to secure a polymer cable in the first thru hole 92, for example, as depicted in the orthopedic screw depicted in FIG. 3.

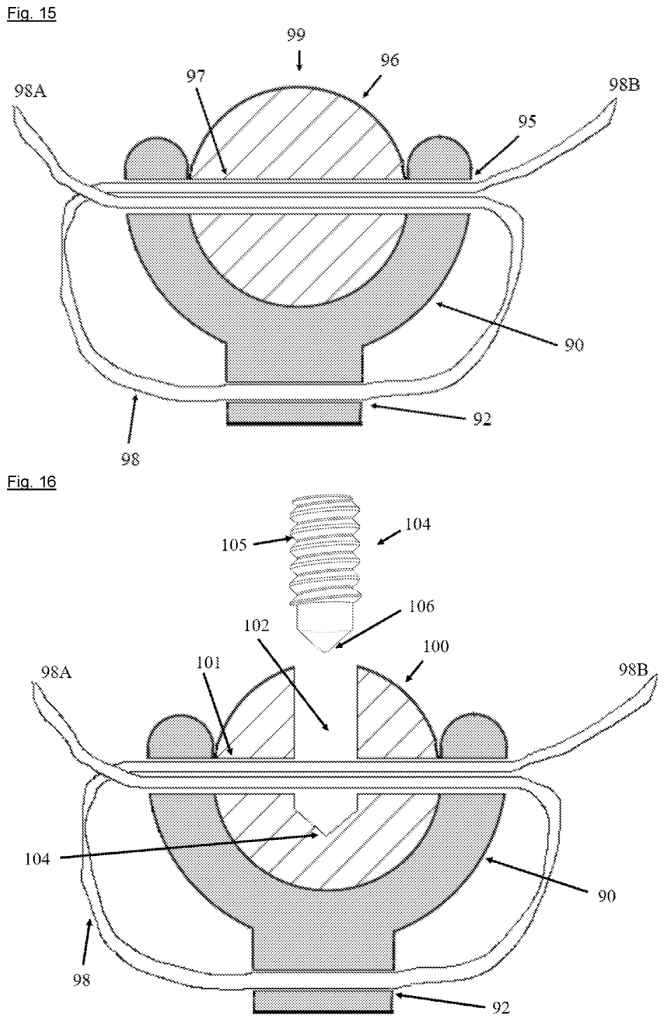

Turning now to FIG. 15, the orthopedic screw of FIG. 14 is depicted supporting a prosthesis 96. The prosthesis 96 sits in cradle 90. Prosthesis 96 comprises a prosthesis thru hole 97. Prosthesis thru hole 97 aligns with second thru hole 95. Polymer cable 98 is positioned in first thru hole 92. First end of polymer cable 98A and second end of polymer cable 98B are passed through opposite sides of second thru hole 95 and prosthesis thru hole 97. Polymer cable 98 can secure prosthesis 96 in cradle 90 by pulling on polymer cable ends 98A and 98B to tighten polymer cable 98 against the orthopedic screw. Subsequently, polymer cable ends 98A and 98B can be tied in a knot, for example, at position 99. Alternative methods of securing the polymer cable to itself at position 99, such as via a button or other fixation device, may be used. The tightening and fixation may be assisted by a fixation assistance device that assists a surgeon with the tightening of the polymer cable. The excess length of polymer cable may then be trimmed.

FIG. 16 depicts an embodiment of a prosthesis 100 positioned in cradle 90. Prosthesis 100 comprises a prosthesis thru hole 101 and a prosthesis threaded hole 102. Prosthesis threaded hole 102 is arranged to receive prosthesis set screw 104. Prosthesis 100 is secured to the orthopedic screw by tightening polymer cable 98 and fully inserting prosthesis set screw 103 into prosthesis threaded hole 102. The prosthesis threaded hole 102 extends at least from an exterior surface of the prosthesis 100 to past the prosthesis thru hole 101. In an embodiment, the center axis of the prosthesis thru hole is perpendicular to the center axis of the prosthesis threaded hole 102. The prosthesis threaded hole 102 comprises an interior thread complimentary to the thread 105 present on the exterior surface of prosthesis set screw 104. The prosthesis threaded hole comprises a prosthesis threaded hole end 103. In an embodiment, the prosthesis threaded hole 102 and prosthesis threaded hole end 103 are complimentary in threading and geometry to prosthesis set screw 104 and prosthesis set screw end 106. In an embodiment, the prosthesis threaded hole does not extend completely through the prosthesis. In an embodiment, the prosthesis threaded hole is a prosthesis blind hole.

In an embodiment, the prosthesis set screw 104 comprises a prosthesis set screw thread 105 that is complimentary to the prosthesis threaded hole thread. In an embodiment, the prosthesis set screw thread 105 is present over less than the entire length of the prosthesis set screw 104. The prosthesis set screw 104 comprises a prosthesis set screw end 106 that is complimentary to the prosthesis threaded hole end 104. Preferably, there is no clearance between the prosthesis threaded hole end 104 and the prosthesis set screw end 106 when the set screw is fully inserted in the prosthesis threaded hole 102. In an embodiment, the prosthesis set screw end is conical. In an embodiment, the prosthesis set screw end is frustoconical. In an embodiment, the prosthesis set screw end is flat. In an embodiment, the prosthesis set screw end is a portion of a spheroid. The set screw may be insertable with a standard screwdriver and can take any suitable screw drive format.

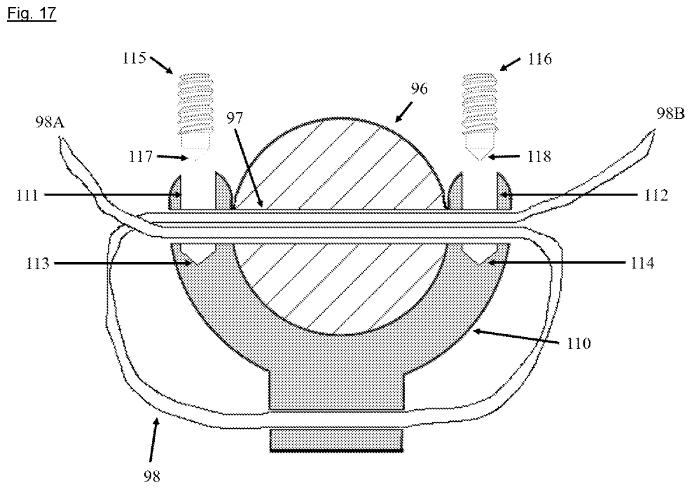

FIG. 17 depicts a cross-section of an embodiment of a prosthesis 96 positioned in the cradle 111 of an orthopedic screw. Prosthesis 96 comprises a prosthesis thru hole or groove 97 extending from one side of prosthesis 96 to the other. The orthopedic screw depicted in FIG. 17 is similar to the orthopedic screw depicted in FIG. 14-16 and may have any of the features described previously. In an embodiment, the orthopedic screw comprises a threaded hole 111 in a wall of the cradle 110. The threaded hole 111 comprises a threaded hole end 113. As depicted in FIG. 17, the orthopedic screw comprises a second threaded hole 112 having a second threaded hole end 114 in a second wall of the cradle 110. The threaded holes 111 and 112 extend from the highest surface of each wall into the cradle 110 and intersect the second thru hole of the orthopedic screw. The threaded holes 111, 112 are associated with set screws 115, 116, respectively. Each set screw 115, 116 comprises a set screw end 117, 118, respectively. The set screw comprises a thread complimentary to the threaded hole thread. The set screw end is complimentary to the respective threaded hole end. In an embodiment, the threaded hole comprises a central axis that is about perpendicular to the central axis of the second thru hole. In an embodiment, the threaded hole comprises a central axis that is about parallel to the longitudinal axis of the orthopedic screw. After fully tightening polymer cable 98 by pulling on ends 98A and 98B, the set screws 115, and 116 may be fully inserted to secure prosthesis 96 in cradle 110. Fixation may be aided by a suitable fixation device. The ends of polymer cable 98 may then be trimmed.

In an embodiment, a prosthesis comprising an elongate member may be secured to an orthopedic screw by positioning a cable in a thru hole of an orthopedic screw, positioning the cable in a thru hole or groove of the prosthesis such that the cable is slidably engaged in the prosthesis, approximating the prosthesis and the orthopedic screw by tightening the cable, and fixing the cable in a tightened state. In an embodiment, the thru hole of the orthopedic screw and the thru hole or groove of a prosthesis have central axes that are substantially parallel. In an embodiment, the cable is positioned in a thru hole of an orthopedic screw such that the cable is slidably engaged in the orthopedic screw. In an embodiment, the step of fixing the cable in a tightened state is performed by tying a knot in the cable. In an embodiment, the step of fixing the cable in a tightened state is performed by tightening a prosthesis set screw, wherein by tightening the prosthesis set screw the set screw is inserted into a prosthesis threaded hole that intersects the prosthesis thru hole, thereby fixing the cable between an end of the set screw and the end of the prosthesis threaded hole.

In an embodiment, a prosthesis comprising an elongate member may be secured to an orthopedic screw comprising a shank and a head, the head comprising a cradle, by the steps of: positioning a cable in a thru hole in the shank of the orthopedic screw such that the cable is engaged in the orthopedic screw, positioning the cable in a first portion of a second thru hole in the cradle of the orthopedic screw, positioning the cable in a thru hole or groove of the prosthesis such that the cable is slidably engaged in the prosthesis, positioning the cable in a second portion of a second thru hole in the cradle of the orthopedic screw, approximating the prosthesis and the orthopedic screw by tightening the cable, and fixing the cable in a tightened state.

In an embodiment, a prosthesis comprising an elongate member may be secured to an orthopedic screw by positioning a cable in a thru hole of an orthopedic screw such that the cable is engaged in the orthopedic screw, placing a first end of the cable in a thru hole or groove of the prosthesis such that the cable is slidably engaged in the prosthesis, placing a second end of the cable in a thru hole or groove of the prosthesis such that the cable is slidably engaged in the prosthesis, approximating the prosthesis and the orthopedic screw by tightening the cable by pulling on the first end and the second end, and fixing the cable in a tightened state. In an embodiment, the cable is in contact with itself in the thru hole or groove of the prosthesis.

In an embodiment, a prosthesis comprising an elongate member may be secured to an orthopedic screw comprising a shank and a head, the head comprising a cradle and a thru hole in the cradle, by the steps of: positioning a cable in a thru hole in the shank of the orthopedic screw such that the cable is engaged in the orthopedic screw, positioning a first end of the cable through a first portion of a second thru hole in the cradle of the orthopedic screw, a thru hole or groove of the prosthesis, and out a second portion of the second thru hole such that the cable is slidably engaged in the prosthesis, positioning a second end of the cable through a second portion of a second thru hole in the cradle of the orthopedic screw, a thru hole or groove of the prosthesis, and out a first portion of the second thru hole such that the cable is slidably engaged in the prosthesis, approximating the prosthesis and the orthopedic screw by tightening the cable by pulling on the first end and the second end, and fixing the cable in a tightened state. In an embodiment, the cable is in contact with itself in the thru hole or groove of the prosthesis and in the second thru hole.

The Examples below further elucidate embodiments of the invention, but of course, should not be construed as in any way limiting the scope of the claims.

### EXAMPLES

### Example 1

Three pedicle screws were evaluated for their ability to retain a polymer cable.

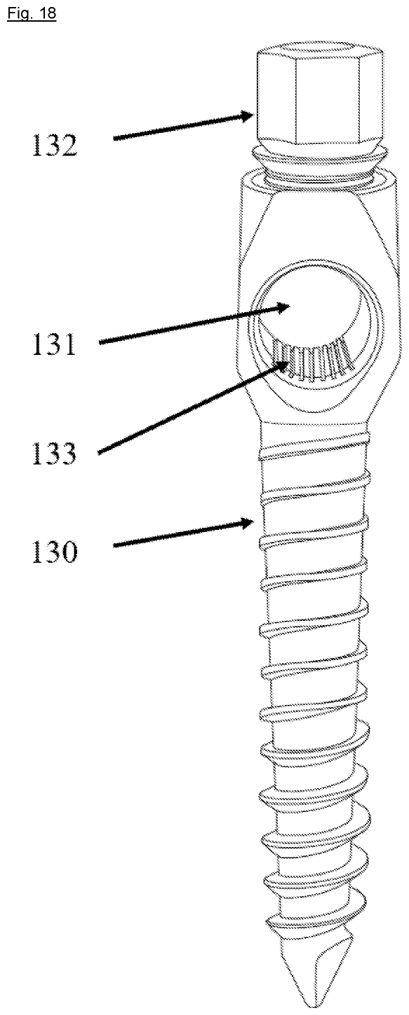

Screw A is shown in FIG. 18. Screw A is a commercial Medtronic TSRH® pedicle screw. Screw A, 130 is not modified from its commercial state. Screw A 130 comprises a hole 131 designed to receive a prosthesis. In operation, a prosthesis is placed in hole 131 and set screw 132 is tightened to fix the prosthesis in hole 131. In order to test Screw A's ability to retain the polymer cable, the polymer cable is placed with its major side in contact with surface 133 and a cylinder having a diameter of 5.5 mm is placed in hole 131 on top of the polymer cable. Set screw 132 is fully tightened to fix the cylinder and polymer cable in Screw A 130.

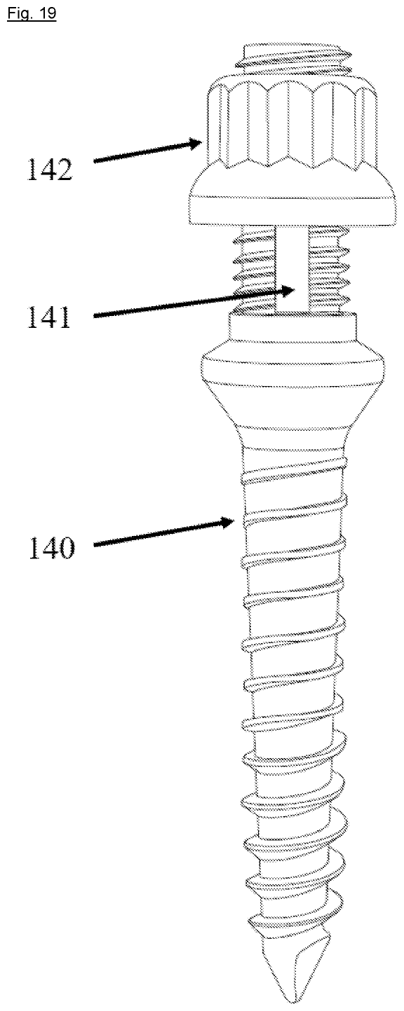

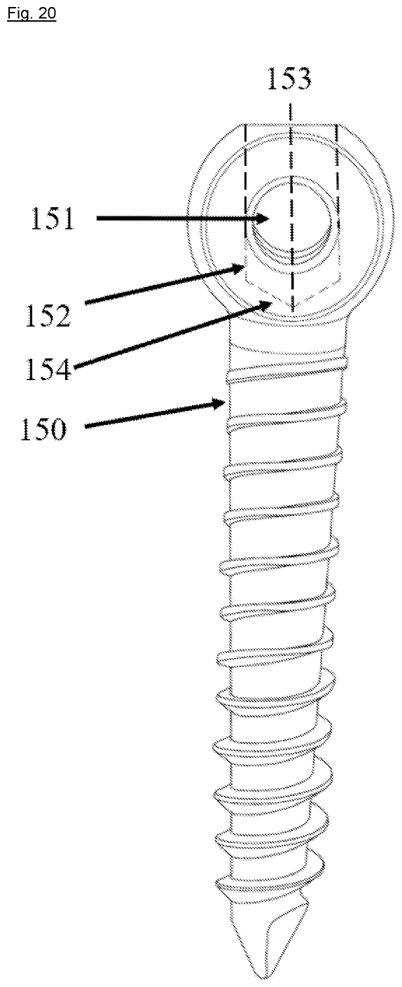

Screw B is shown in FIG. 19. Screw B is a modified version of a commercial Acromed DePuy TiMX® pedicle screw. Screw B 140 is modified from its commercial state to contain thru hole 141. Thru hole 141 has a width of 5 mm. A polymer cable is placed in slot 141 and nut 142 fully tightened to fix the polymer cable to Screw B 140.

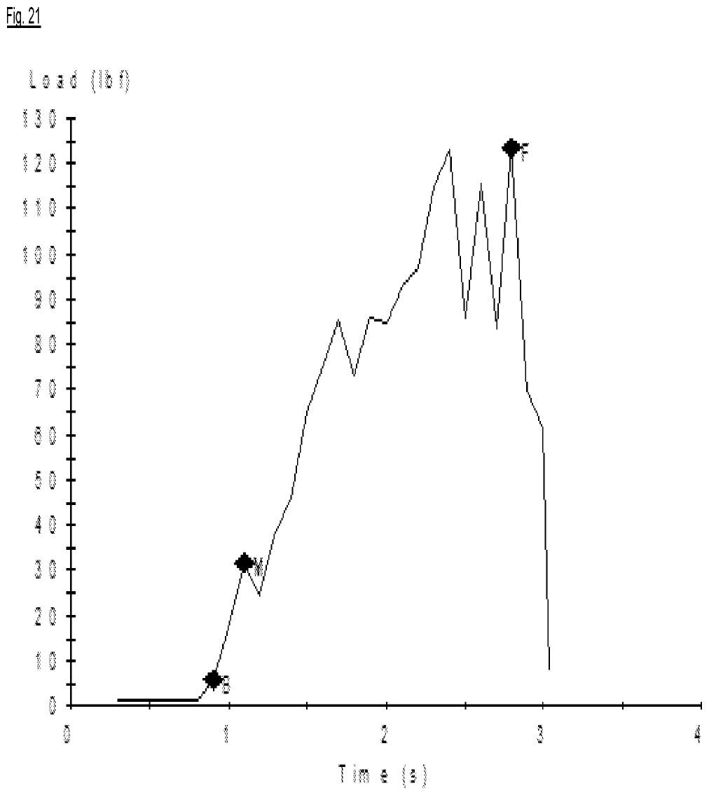

Screw C is shown in FIG. 20. Screw C is a modified version of a commercial pedicle screw from Zimmer Biomet, model PROTASUL®—100 titanium. Screw C 150 comprises thru hole 151. Screw C is modified from its commercial form by drilling a threaded hole 152 having central axis 153. Central axis 153 is coaxial with the longitudinal axis of Screw C. Threaded hole 152 comprises threaded hole end 154 having a conical shape. Threaded hole 152 has a diameter of 5 mm. A set screw (not pictured) having a shape generally in accordance with the set screw depicted in FIG. 2 and an exterior thread complimentary to threaded hole 152 is insertable in threaded hole 152. The set screw end corresponds to threaded hole end 154. A polymer cable is placed in thru hole 152 and the set screw fully tightened to fix polymer cable to Set Screw C 150.

The pull-out forces of the polymer cables fixed in Screw A, Screw B, and Screw C are tested as follows. The polymer cable tested is a Dyneema® Purity Radiopaque Cerclage Cable available from DSM Biomedical, Inc. The polymer cable has a width of approximately 4 mm and a thickness of approximately 0.5 mm. A 25 cm length of the polymer cable is provided. A first portion of the cable is placed in the opening (the hole 131 of Screw A, in the slot 141 of screw B, and in the thru hole 151 of Screw C, as appropriate) and a 12 cm long loop created by passing an end of the polymer cable back through the opening and tightening the polymer cable in the screw. The polymer cable thus touches itself when it is positioned in the thru hole. The set screws of Screw A and Screw C and the nut of Screw B are tightened to the approximate torque value indicated in Table 1.

An MTS QTest/1L Elite Controller is used to test the pull-out force. The screw shank is held horizontally in a stationary first clamp while the loop of the polymer cable is looped over a rod that is held in a movable second clamp. The results are shown in the below Table 1.

TABLE 1
Example 1 Results

| Screw | Set Screw Torque (Nm) | Maximum Force (N) |
|---|---|---|
| A | 10.8 | 534 |
| B | 13.6 | 289 |
| C | 13.6 | 778+ (load cell max) |

Screw A reached a pull-out force of about 515 N before pull-out of the polymer cable is observed. Inspection of the polymer cable post test revealed flattening of the cable, some deformation/compression of the cable and striations that match the machined ridges inside the hole on surface 133. During the test there is some slippage of the cable before ultimate breakage occurs. A plot of load (lbf) vs. time (s) is shown in FIG. 21. The test is performed three times with little variation in results noted between individual tests. It is speculated that the series of machined ridges increase the pull-out force relative to a screw having a smooth surface in their place.

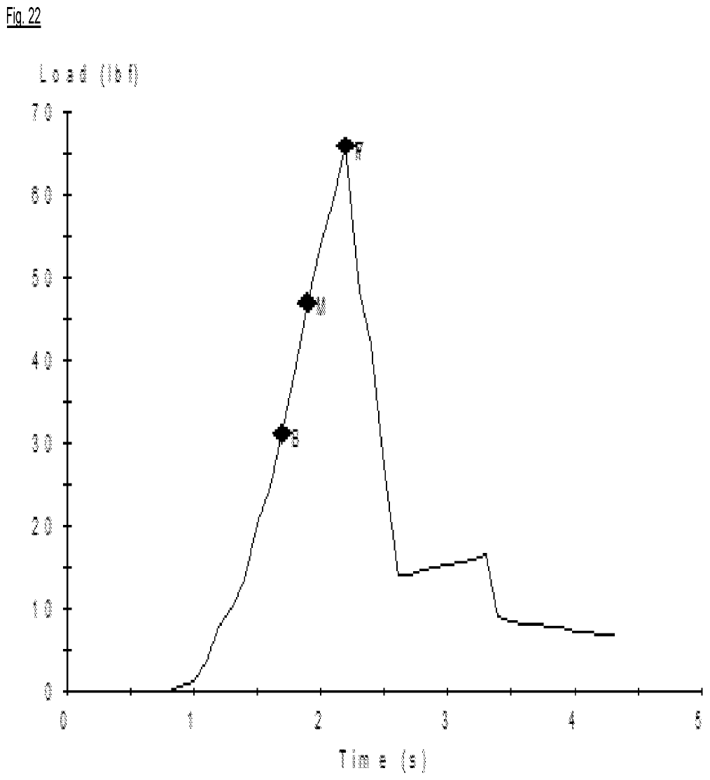

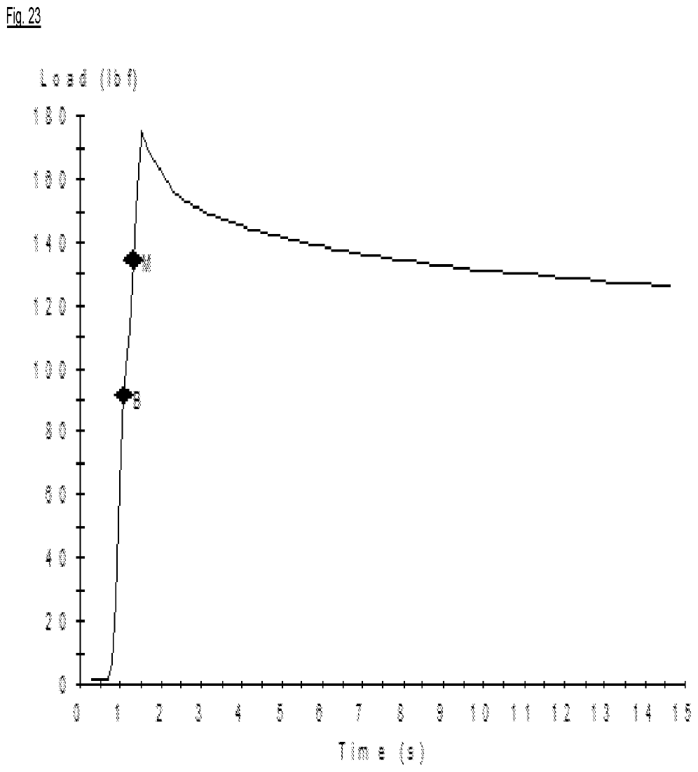

Screw B reaches a pull-out force of about 260 N. A plot of load (lbf) vs. time (s) is shown in FIG. 22. The polymer cable ruptures and clear damage to the polymer cable is observed. Shearing of the cable against surfaces of Screw B is presumed the cause for rupture. The test is performed once and not repeated.

Screw C reached a pull-out force of 750 N without breakage or pull-out. Inspection of the cable post-test reveals flattening of the cable, some deformation/compression of the cable and localized damage to a small number of the filaments relative to the overall construction of the polymer cable. There is an imprint corresponding to the shape of the set screw end and threaded hole end visible in the polymer cable. No slippage or movement of the polymer cable occurs. This is confirmed by the plot in FIG. 23. This test is performed five times and consistent results are observed.

### Additional Description of Exemplary Embodiments

1. An orthopedic screw configured to retain a polymer cable when the polymer cable is positioned in a thru hole present in the body of said orthopedic screw.
2. The orthopedic screw according to any one of the previous exemplary embodiments, wherein the orthopedic screw is configured to secure the polymer cable in the thru hole.
3. The orthopedic screw according to any one of the previous exemplary embodiments, further comprising a set screw and a complimentary threaded hole, wherein the threaded hole intersects the thru hole and wherein the polymer cable is secured in the thru hole by tightening the set screw.
4. The orthopedic screw according to any one of the previous exemplary embodiments, further comprising a set screw and a complimentary threaded hole, wherein the threaded hole intersects the thru hole and extends beyond the thru hole, and wherein the polymer cable is secured in the thru hole by tightening the set screw.
5. The orthopedic screw according to any one of the previous exemplary embodiments, wherein the threaded hole comprises a threaded hole end and the set screw comprises a set screw end, and where set screw end is complimentary to the threaded hole end.
6. The orthopedic screw according to any one of the previous exemplary embodiments, further comprising a cradle.
7. An orthopedic screw comprising:
   a. a screw body comprising
      i. a head;
      ii. a shank connected to the head and comprising an exterior thread, the shank having a longitudinal axis,
      iii. a thru hole extending from one side of the screw body to the other and through the longitudinal axis, and
      iv. a threaded hole intersecting the thru hole, and extending at least from an exterior surface of the head to past the thru hole, the threaded hole comprising a threaded hole end and a threaded hole thread, and
   b. a set screw comprising a set screw thread, the set screw thread being complimentary to the threaded hole thread, and the set screw comprising a set screw end complimentary to the threaded hole end.
8. The orthopedic screw according to any one of the previous exemplary embodiments, further comprising:
v. a cradle forming at least part of the head, the cradle comprising a base and a wall, the cradle being adapted to support an elongate member, and wherein the threaded hole extends at least from the base of the cradle to past the thru hole.
9. The orthopedic screw according to the previous exemplary embodiment, further comprising:
   vi. a second thru hole in the wall of the cradle and extending through the cradle.
10. An orthopedic screw comprising:
   a. a screw body comprising
      i. a head;
      ii. a shank connected to the head and comprising an exterior thread, the shank having a longitudinal axis,
      iii. a thru hole extending from one side of the shank to the other and through the longitudinal axis,
      iv. a cradle forming at least part of the head, the cradle comprising a base and a wall, the cradle being adapted to support an elongate member,
      v. a second thru hole in the wall of the cradle and extending through the cradle, and
      vi. a threaded hole comprising a threaded hole end, the threaded hole present in the wall of the cradle, wherein the threaded hole intersects the second thru hole, and
   b. a set screw comprising a set screw end, the set screw comprising a set screw thread, the set screw thread being complimentary to the threaded hole thread, and the set screw comprising a set screw end complimentary to the threaded hole end.
11. The orthopedic screw according to any one of the previous exemplary embodiments, wherein the set screw end and the threaded hole end are in contact when the set screw is fully inserted into the threaded hole.
12. The orthopedic screw according to any one of the previous exemplary embodiments, wherein the threaded hole extends in the direction of the longitudinal axis.
13. The orthopedic screw according to any one of the previous exemplary embodiments, wherein the central axis of the threaded hole is about parallel to the longitudinal axis.
14. The orthopedic screw according to any one of the previous exemplary embodiments, further comprising a cradle forming at least part of the head, the cradle comprising a base and a wall, the cradle being adapted to support an elongate member, and wherein the threaded hole extends at least from the base of the cradle to past the thru hole.
15. An orthopedic screw comprising:
   a. a screw body comprising
      i. a head;
      ii. a shank connected to the head and comprising an exterior thread, the shank having a longitudinal axis,
      iii. a thru hole extending from one side of the shank to the other and through the longitudinal axis,
      iv. a cradle forming at least part of the head, the cradle comprising a base and a wall, the cradle being adapted to support an elongate member, and
      v. a second thru hole in the wall of the cradle and extending through the cradle.
16. The orthopedic screw according to any one of the previous exemplary embodiments, wherein the orthopedic screw is a mono-axial screw.
17. The orthopedic screw according to any one of the previous exemplary embodiments, wherein the orthopedic screw is a poly-axial screw.
18. The orthopedic screw according to any one of the previous exemplary embodiments, wherein the threaded hole extends at least 0.1 mm, 0.5 mm, 1.0 mm, 1.5 mm, 2 mm, 2.5 mm, 3.0 mm, 3.5 mm, 4.0 mm, 4.5 mm, or 5.0 mm past the thru hole.
19. The orthopedic screw according to any one of the previous exemplary embodiments, wherein the threaded hole extends at most 10 mm, 9 mm, 8 mm, 7.5 mm, 7 mm, 6.5 mm, 6 mm, 5.5 mm. 5 mm, 4.5 mm or 4 mm past the thru hole.
20. The orthopedic screw according to any one of the previous exemplary embodiments, wherein the head and shank are formed as a single piece.
21. The orthopedic screw according to any one of the previous exemplary embodiments, wherein the cradle and shank are formed as a single piece.
22. The orthopedic screw according to any one of the previous exemplary embodiments, wherein the cradle comprises two walls.
23. The orthopedic screw according to any one of the previous exemplary embodiments, wherein the cradle is open at its top.
24. The orthopedic screw according to any one of the previous exemplary embodiments, wherein the cradle is closed at its top, such that an elongate member may not be inserted into the cradle by moving a portion of the elongate member in the direction of the longitudinal axis.
25. The orthopedic screw according to any one of the previous exemplary embodiments, wherein the wall further comprises a second thru hole.
26. The orthopedic screw according to any one of the previous exemplary embodiments, wherein the cradle comprises two walls and each wall comprises a wall thru hole.
27. The orthopedic screw according to any one of the previous exemplary embodiments, further comprising a second wall, wherein the second wall comprises a second threaded hole and a corresponding second set screw.
28. The orthopedic screw according to any one of the previous exemplary embodiments, further comprising a second wall and a second threaded hole comprising a second threaded hole end, the second threaded hole present in the second wall of the cradle, wherein the second threaded hole intersects the second thru hole, and a second set screw comprising a second set screw end, the second set screw comprising a second set screw thread, the second set screw thread being complimentary to the second threaded hole thread, and the second set screw comprising a second set screw end complimentary to the second threaded hole end.
29. The orthopedic screw according to any one of the previous exemplary embodiments, wherein the cradle comprises two walls and each wall comprises a wall threaded hole and a corresponding wall set screw.
30. The orthopedic screw according to any one of the previous exemplary embodiments, wherein the cradle is adapted to support a cylindrical elongate member.
31. The orthopedic screw according to any one of the previous exemplary embodiments, wherein the cradle is substantially semicircular.
32. The orthopedic screw according to any one of the previous exemplary embodiments, wherein the thru hole is circular.
33. The orthopedic screw according to any one of the previous exemplary embodiments, wherein the thru hole is rectangular.
34. The orthopedic screw according to any one of the previous exemplary embodiments, wherein the thru hole is trapezoidal.
35. The orthopedic screw according to any one of the previous exemplary embodiments, wherein the thru hole comprises a central axis that is transverse to the longitudinal axis.
36. The orthopedic screw according to any one of the previous exemplary embodiments, wherein the thru hole is located between the base of the head and the start of the exterior thread.
37. The orthopedic screw according to any one of the previous exemplary embodiments, wherein the thru hole is located between the base of the cradle and the start of the exterior thread.
38. The orthopedic screw according to any one of the previous exemplary embodiments, wherein the thru hole is adapted to receive a polymer cable.
39. The orthopedic screw according to any one of the previous exemplary embodiments, wherein the threaded hole comprises a threaded hole thread over less than its entire length.
40. The orthopedic screw according to any one of the previous exemplary embodiments, wherein the threaded hole end is conical.
41. The orthopedic screw according to any one of the previous exemplary embodiments, wherein the threaded hole end is frustoconical.
42. The orthopedic screw according to any one of the previous exemplary embodiments, wherein the threaded hole extends past the thru hole by at least 1 mm.
43. The orthopedic screw according to any one of the previous exemplary embodiments, wherein the threaded hole is coaxial with the longitudinal axis.
44. The orthopedic screw according to any one of the previous exemplary embodiments, wherein the set screw thread extends over less than the entire length of the set screw.
45. The orthopedic screw according to any one of the previous exemplary embodiments, wherein the threaded hole is a blind hole.
46. The orthopedic screw according to any one of the previous exemplary embodiments, wherein the threaded hole does not extend completely through the screw body.
47. The orthopedic screw according to any one of the previous exemplary embodiments, wherein the set screw end is conical.
48. The orthopedic screw according to any one of the previous exemplary embodiments, wherein the set screw end is frustoconical.
49. The orthopedic screw according to any one of the previous exemplary embodiments, wherein the set screw end comprises a piercing element arranged to pierce a polymer cable present in the thru hole.
50. The orthopedic screw according to any one of the previous exemplary embodiments, wherein the set screw end comprises a plurality of piercing elements arranged to pierce a polymer cable present in the thru hole.
51. The orthopedic screw according to any one of the previous exemplary embodiments, wherein the threaded hole end comprises a receptacle for a piercing element present on the set screw end.
52. The orthopedic screw according to any one of the previous exemplary embodiments, wherein the center axis of the thru hole is orthogonal to the center axis of the threaded hole.
53. The orthopedic screw according to any one of the previous exemplary embodiments, wherein the cradle further comprises a second thru hole.
54. The orthopedic screw according to any one of the preceding exemplary embodiments, wherein the thru hole, the threaded hole, and the set screw are configured to retain a polymer cable positioned in the thru hole.
55. The orthopedic screw according to any one of the previous exemplary embodiments, wherein the second thru hole is circular.
56. The orthopedic screw according to any one of the previous exemplary embodiments, wherein the second thru hole is rectangular.
57. The orthopedic screw according to any one of the previous exemplary embodiments, wherein the second thru hole is trapezoidal.
58. The orthopedic screw according to any one of the previous exemplary embodiments, wherein the second thru hole comprises a central axis that is transverse to the longitudinal axis.
59. The orthopedic screw according to any one of the previous exemplary embodiments, wherein the thru hole comprises a central axis and the second thru hole comprises a second central axis, and the central axis and the second central axis are substantially parallel.
60. The orthopedic screw according to any one of the previous exemplary embodiments, wherein the thru hole comprises a central axis and the second thru hole comprises a second central axis, and the central axis and the second central axis are not substantially parallel.
61. The orthopedic screw according to any one of the previous exemplary embodiments, wherein the thru hole comprises a central axis and the second thru hole comprises a second central axis, and the central axis and the second central axis are substantially perpendicular.
62. The orthopedic screw according to any one of the previous exemplary embodiments, wherein the second thru hole is located in the half of the cradle that is furthest away from the base of the cradle along the longitudinal axis.
63. The orthopedic screw according to any one of the previous exemplary embodiments, wherein the second thru hole is adapted to receive a polymer cable.
64. The orthopedic screw according to any one of the preceding exemplary embodiments, wherein when an elongate member is positioned in the cradle, the elongate member may move in the direction of the longitudinal axis of the orthopedic screw or in the axial direction of the elongate member, but may not substantially move laterally due to the one or more walls of the cradle.
65. The orthopedic screw according to any one of the previous exemplary embodiments, wherein when an elongate member is present in the cradle, and when a cable is present in the second thru hole and tightened, the cable exerts a force on the elongate member in the direction of the longitudinal axis.
66. A method of securing a body part of a patient comprising the steps of:
   a. securing an orthopedic screw to a first body part of a patient,
   b. securing the polymer cable to s second body part of a patient, and
   c. securing the polymer cable in the orthopedic screw.
67. A method for stabilizing the spine of a mammal comprising the steps of:
   a. inserting a plurality of orthopedic screws into the spine of a patient, the orthopedic screws being configured for securing a polymer cable and comprising a head adapted to support an elongate member,
   b. securing one or more polymer cables in the plurality of orthopedic screws,
   c. providing a spinal prosthesis comprising one or more elongate members,
   d. placing the one or more elongate members proximate the heads of the plurality of orthopedic screws,
   e. fixing the one or more elongate members in place relative to the plurality of orthopedic screws with the one or more polymer cables.
68. The method of any one of the preceding exemplary embodiments, wherein the step of securing the one or more polymer cables comprises the steps of:
   b1. inserting a polymer cable through a thru hole in the orthopedic screw, wherein the thru hole runs in a direction about transverse to a longitudinal axis of the orthopedic screw, and
   b2. inserting a set screw into a complimentary threaded hole on the orthopedic screw, wherein the threaded hole intersects and extends beyond the thru hole, thereby securing the polymer cable in the orthopedic screw.
69. A method of securing a prosthesis comprising the steps of:
   a. securing a polymer cable in an orthopedic screw, and
   b. securing a medical prosthesis to the orthopedic screw with the polymer cable.
70. A method of securing a prosthesis comprising the steps of:
   a. securing a polymer cable in an orthopedic screw, and
   b. securing a medical prosthesis to the orthopedic screw with the polymer cable.
71. A method of securing a prosthesis comprising the steps of:
   a. securing a polymer cable in an orthopedic screw by positioning the polymer cable in a thru hole of the orthopedic screw and tightening a set screw, and
   b. securing a medical prosthesis to the orthopedic screw with the polymer cable.
72. A method of securing a prosthesis comprising the steps of:
   a. securing a polymer cable in an orthopedic screw by positioning the polymer cable in a thru hole of the orthopedic screw and tightening a set screw, and
   b. securing a medical prosthesis to the orthopedic screw with the polymer cable.
73. The method of any one of the previous exemplary embodiments, further comprising the step of inserting the orthopedic screw into a bone of a patient.
74. The method of any one of the previous exemplary embodiments, further comprising the steps of: aligning a second thru hole on the orthopedic screw with a thru hole on the medical prosthesis, and passing the polymer cable through the second thru hole on the orthopedic screw and the thru hole on the medical prosthesis.
75. The method of any one of the previous exemplary embodiments, further comprising the steps of: aligning a second thru hole on the orthopedic screw with a thru hole on the medical prosthesis, and passing the polymer cable through both the second thru hole on the orthopedic screw and the thru hole on the medical prosthesis, wherein the second thru hole is present on a cradle of the orthopedic screw.
76. The method of any one of the previous exemplary embodiments, further comprising the steps of: aligning a second thru hole on the orthopedic screw with a thru hole on the medical prosthesis, passing the polymer cable through the second thru hole on the orthopedic screw and the thru hole on the medical prosthesis, and securing the polymer cable in the thru hole of the medical prosthesis.
77. The method of any one of the previous exemplary embodiments, wherein the step of securing a medical prosthesis to the orthopedic screw is performed by tying the polymer cable to the medical prosthesis.
78. The method of any one of the previous exemplary embodiments, wherein the step of securing a medical prosthesis to the orthopedic screw is performed by tying the polymer cable to the medical prosthesis.
79. A kit comprising:
   a. a plurality of orthopedic screws according to any one of the preceding exemplary embodiments,
   b. a spinal prosthesis comprising an elongate member, and
   c. a plurality of polymer cables.
80. The method or kit according to any one of the preceding exemplary embodiments, wherein the spinal prosthesis is a cylinder.
81. The method or kit according to any one of the preceding exemplary embodiments, wherein the spinal prosthesis comprises a plurality of elongate members extending from a central body.
82. The method or kit according to any one of the preceding exemplary embodiments, wherein the elongate member comprises a plurality of ridges, slots, or notches.
83. The method or kit according to any one of the preceding exemplary embodiments, wherein the elongate member is configured to engage the orthopedic screw and thereby reduce axial translation of the elongate member relative to the orthopedic screw.
84. The method or kit according to any one of the preceding exemplary embodiments, wherein the spinal prosthesis comprises a plurality of thru holes.
85. The method or kit according to any one of the preceding exemplary embodiments, wherein the spinal prosthesis comprises a plurality of thru holes and accompanying set screws for securing the polymer cable in one or more thru holes of the spinal prosthesis.
86. A spinal prosthesis comprising:
   a. an elongate member having a member longitudinal axis,
   b. a prosthesis thru hole extending from one side of the elongate member to the other and intersecting a plane containing the member longitudinal axis,
   c. a prosthesis threaded hole intersecting the prosthesis thru hole, the prosthesis threaded hole comprising a prosthesis threaded hole thread and extending at least from a surface of the elongate member to past the thru hole, and
   d. a prosthesis set screw comprising a prosthesis set screw thread, the prosthesis set screw thread being complimentary to the prosthesis threaded hole thread.
87. The spinal prosthesis according to any one of the preceding exemplary embodiments, wherein the prosthesis threaded hole is a blind hole.
88. The spinal prosthesis according to any one of the preceding exemplary embodiments, wherein the prosthesis threaded hole does not extend completely through the elongate member.
89. The spinal prosthesis according to any one of the preceding exemplary embodiments wherein the member longitudinal axis intersects the axis of the prosthesis thru hole.
90. The spinal prosthesis according to any one of the preceding exemplary embodiments wherein the member longitudinal axis intersects the axis of the prosthesis threaded hole.
91. The spinal prosthesis according to any one of the preceding exemplary embodiments wherein the member longitudinal axis intersects the axis of the prosthesis thru hole and the axis of the prosthesis threaded hole.
92. The spinal prosthesis according to any one of the preceding exemplary embodiments, wherein the axis of the prosthesis thru hole intersects the axis of the prosthesis threaded hole.
93. The spinal prosthesis according to any one of the preceding exemplary embodiments, wherein the prosthesis threaded hole comprises a prosthesis threaded hole end.
94. The spinal prosthesis according to any one of the preceding exemplary embodiments, wherein the prosthesis threaded hole comprises a prosthesis threaded hole end, the prosthesis set screw comprises a prosthesis set screw end, and the prosthesis set screw end is complimentary to the prosthesis threaded hole end.
95. The spinal prosthesis according to any one of the preceding exemplary embodiments, wherein the prosthesis thru hole, prosthesis threaded hole, and prosthesis set screw are configured to retain a polymer cable positioned in the prosthesis thru hole.
96. The spinal prosthesis according to any one of the preceding exemplary embodiments, wherein the prosthesis set screw end and the prosthesis threaded hole end are in contact when the prosthesis set screw is fully inserted into the prosthesis threaded hole.
97. A method of securing a prosthesis comprising an elongate member to an orthopedic screw comprising the steps of:
   a. positioning a cable in a thru hole of an orthopedic screw such that the cable is engaged in the orthopedic screw,
   b. positioning the cable in a thru hole or groove of the prosthesis such that the cable is slidably engaged in the prosthesis,
   c. approximating the prosthesis and the orthopedic screw by tightening the cable, and
   d. fixing the cable in a tightened state.
98. A method of securing a prosthesis comprising an elongate member to an orthopedic screw comprising a shank and a head, the head comprising a cradle, comprising the steps of:
   a. positioning a cable in a thru hole in the shank of the orthopedic screw such that the cable is engaged in the orthopedic screw,
   b. positioning the cable in a first portion of a second thru hole in the cradle of the orthopedic screw,
   c. positioning the cable in a thru hole or groove of the prosthesis such that the cable is slidably engaged in the prosthesis,
   d. positioning the cable in a second portion of a second thru hole in the cradle of the orthopedic screw,
   e. approximating the prosthesis and the orthopedic screw by tightening the cable, and
   f. fixing the cable in a tightened state.
99. A method of securing a prosthesis comprising an elongate member to an orthopedic screw comprising the steps of:
   a. positioning a cable in a thru hole of an orthopedic screw such that the cable is engaged in the orthopedic screw,
   b. passing a first end of the cable through a thru hole or groove of the prosthesis in a first direction such that the cable is slidably engaged in the prosthesis,
   c. placing a second end of the cable in the thru hole or groove of the prosthesis in a second direction, opposite of the first direction, such that the cable is slidably engaged in the prosthesis,
   d. approximating the prosthesis and the orthopedic screw by tightening the cable by pulling on the first end and the second end, and
   e. fixing the cable in a tightened state.
100. A method of securing a prosthesis comprising an elongate member to an orthopedic screw comprising a shank and a head, the head comprising a cradle and a thru hole in the cradle, comprising the steps of:
   a. positioning a cable in a thru hole in the shank of the orthopedic screw such that the cable is engaged in the orthopedic screw,
   b. positioning a first end of the cable through a first portion of a second thru hole in the cradle of the orthopedic screw, through a thru hole or groove of the prosthesis, and out a second portion of the second thru hole such that the cable is slidably engaged in the prosthesis,
   c. positioning a second end of the cable through a second portion of a second thru hole in the cradle of the orthopedic screw, through a thru hole or groove of the prosthesis, and out a first portion of the second thru hole such that the cable is slidably engaged in the prosthesis,
   d. approximating the prosthesis and the orthopedic screw by tightening the cable by pulling on the first end and the second end, and
   e. fixing the cable in a tightened state.
101. The method according to any one of the preceding exemplary embodiments, wherein the thru hole of the orthopedic screw and the thru hole or groove of a prosthesis have central axes that are substantially parallel after fully tightening the cable.
102. The method according to any one of the preceding exemplary embodiments, wherein the thru hole of the orthopedic screw, the thru hole or groove of the prosthesis, and the second thru hole have central axes that are substantially parallel after fully tightening the cable.
103. The method according to any one of the preceding exemplary embodiments, wherein the cable is positioned in the thru hole of an orthopedic screw such that the cable is slidably engaged in the orthopedic screw.
104. The method according to any one of the preceding exemplary embodiments, wherein the step of fixing the cable in a tightened state is performed by tying a knot in the cable.
105. The method according to any one of the preceding exemplary embodiments, wherein the step of fixing the cable in a tightened state is performed by tightening a prosthesis set screw, wherein by tightening the prosthesis set screw the prosthesis set screw is inserted into a prosthesis threaded hole that intersects the prosthesis thru hole, thereby fixing the cable between an end of the set screw and the end of the prosthesis threaded hole.
106. The method according to any one of the preceding exemplary embodiments, wherein the cable is in contact with itself in the thru hole or groove of the prosthesis.
107. The method according to any one of the preceding exemplary embodiments, wherein the cable is in contact with itself in the second thru hole.
108. A fixation system comprising
   a. the orthopedic screw according to any one of the previous exemplary embodiments,
   b. a polymer cable arranged in the thru hole.
109. A fixation system comprising:
   a. the spinal prosthesis according to any one of the previous exemplary embodiments,
   b. a polymer cable arranged in the prosthesis thru hole.
110. The fixation system, kit, or method of any one of the previous exemplary embodiments, wherein the cable is a polymer cable.
111. The fixation system, kit, or method of any one of the previous exemplary embodiments, wherein the polymer cable has a substantially rectangular cross-section.
112. The fixation system, kit, or method of any one of the previous exemplary embodiments, wherein the polymer cable comprises polyethylene.
113. The fixation system, kit, or method of any one of the previous exemplary embodiments, wherein the polymer cable comprises polymer fibers.
114. The fixation system, kit, or method of any one of the previous exemplary embodiments, wherein the polymer cable consists of polymer fibers.
115. The fixation system, kit, or method of any one of the previous exemplary embodiments, wherein the polymer cable comprises a braid or a yarn.
116. The fixation system, kit, or method of any one of the previous exemplary embodiments, wherein the polymer cable comprises a braid of polymer fibers.
117. The fixation system, kit, or method of any one of the previous exemplary embodiments, wherein the polymer cable comprises a braid of polyethylene fibers.
118. The fixation system, kit, or method of any one of the previous exemplary embodiments, wherein the polymer cable comprises a braid of polymer cables.
119. The fixation system, kit, or method of any of the previous exemplary embodiments, wherein the polymer cable comprises a braid that comprises a hollow tubular braid, a solid circular braid, a spiroid braid, a flat braid, a core-sheath braid, or a braid-on-braid.
120. The fixation system, kit, or method of any one of the previous exemplary embodiments, wherein the polymer cable comprises a yarn.
121. The fixation system, kit, or method of any one of the previous exemplary embodiments, wherein the polymer cable comprises a braided, knitted, or woven cable, wherein the cable comprises polymer fibers.
122. The fixation system, kit, or method of any one of the previous exemplary embodiments, wherein the polymer cable comprises a monofilament or a multifilament yarn.
123. The fixation system, kit, or method of any one of the previous exemplary embodiments, wherein the polymer cable comprises a radiopaque agent.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. While certain optional features are described as embodiments of the invention, the description is meant to encompass and specifically disclose all combinations of these embodiments unless specifically indicated otherwise or physically impossible.

1. An orthopedic screw configured to retain a polymer cable when the polymer cable is positioned in a thru hole present in the body of said orthopedic screw, comprising a set screw and a complimentary threaded hole, wherein the threaded hole intersects the thru hole and extends beyond the thru hole, and wherein the polymer cable may be secured in the thru hole by tightening the set screw.

2. (canceled)

3. (canceled)

4. The orthopedic screw according to claim 1, wherein the threaded hole comprises a threaded hole end and the set screw comprises a set screw end, and wherein the set screw end is complimentary to the threaded hole end.

5. The orthopedic screw according to claim 1, further comprising a cradle.

6. An orthopedic screw comprising:
a. a screw body comprising
i. a head;
ii. a shank connected to the head and comprising an exterior thread, the shank having a longitudinal axis,
iii. a thru hole extending from one side of the screw body to the other and through the longitudinal axis, and
iv. a threaded hole intersecting the thru hole, and extending at least from an exterior surface of the head to past the thru hole, the threaded hole comprising
a threaded hole end and a threaded hole thread, and
b. a set screw comprising a set screw thread, the set screw thread being complimentary to the threaded hole thread, and the set screw comprising a set screw end complimentary to the threaded hole end.

7. The orthopedic screw according to claim 6, wherein the threaded hole extends in the direction of the longitudinal axis.

8. The orthopedic screw according to claim 7, wherein a central axis of the threaded hole is about parallel to the longitudinal axis.

9. The orthopedic screw according to claim 6, wherein the orthopedic screw is a mono-axial screw.

10. The orthopedic screw according to claim 6, wherein the orthopedic screw is a poly-axial screw.

11. The orthopedic screw according to claim 6, wherein the threaded hole extends at least 1.0 mm past the thru hole.

12. The orthopedic screw according to claim 6, further comprising:
v. a cradle forming at least part of the head, the cradle comprising a base and a wall, the cradle being adapted to support an elongate member, and wherein the threaded hole extends at least from the base of the cradle to past the thru hole.

13. The orthopedic screw according to claim 12, further comprising:
vi. a second thru hole in the wall of the cradle and extending through the cradle.

14. The orthopedic screw according to claim 6, wherein the threaded hole comprises a threaded hole thread over less than its entire length.

15. The orthopedic screw according to claim 6, wherein the threaded hole end is frustoconical.

16. The orthopedic screw according to claim 6, wherein the set screw thread extends over less than the entire length of the set screw.

17. The orthopedic screw according to claim 15, wherein the set screw end is frustoconical.

18. A fixation system comprising
a. the orthopedic screw according to claim 6,
b. a polymer cable arranged in the thru hole.

19. The fixation system of claim 18, wherein the polymer cable comprises a braided, knitted, or woven cable, wherein the cable comprises polymer fibers.

20. (canceled)

21. A method of securing a prosthesis comprising an elongate member to an orthopedic screw comprising a shank and a head, the head comprising a cradle and a thru hole in the cradle, comprising the steps of:
a. positioning a cable in a thru hole in the shank of the orthopedic screw such that the cable is engaged in the orthopedic screw,
b. positioning a first end of the cable through a first portion of a second thru hole in the cradle of the orthopedic screw, through a thru hole or groove of the prosthesis, and
out a second portion of the second thru hole such that the cable is slidably engaged in the prosthesis,
c. positioning a second end of the cable through a second portion of a second thru hole in the cradle of the orthopedic screw, through a thru hole or groove of the prosthesis, and out a first portion of the second thru hole such that the cable is slidably engaged in the prosthesis,
d. approximating the prosthesis and the orthopedic screw by tightening the cable by pulling on the first end and the second end, and
e. fixing the cable in a tightened state.

22. The method of claim 21 or 22, wherein the cable comprises a braided, knitted, or woven cable, wherein the cable comprises polymer fibers.

23. (canceled)

24. The method of claim 21, wherein the thru hole of the orthopedic screw and the thru hole or groove of a prosthesis have central axes that are substantially parallel after tightening the cable.
